(19) United States
(12) Patent Application Publication
(10) Pub. No.: US 2006/0246061 A1
(43) Pub. Date: Nov. 2, 2006

(54) Method for generating monoclonal antibodies

(76) Inventors: M. Lamine MBow, King of Prussia, PA (US); Patrick Branigan, Lansdowne, PA (US); Jill Giles-Komar, Downingtown, PA (US); Linda Snyder, Pottstown, PA (US); George Heavner, Malvern, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/477,036

(22) Filed: Jun. 28, 2006

Related U.S. Application Data
(63) Continuation of application No. 10/600,348, filed on Jun. 20, 2003, now abandoned.
(60) Provisional application No. 60/390,498, filed on Jun. 21, 2002.

(51) Int. Cl. A61K 39/395; C12P 21/04

(52) U.S. Cl. 424/141.1; 435/70.21; 800/6

(57) ABSTRACT

Methods for generating monoclonal antibodies in Th1-biased rodents are disclosed. The monoclonal antibodies are useful as therapeutic agents, diagnostic agents or research reagents.

### CROSS-REFERENCE TO RELATED APPLICATION

This application claims the benefit of U.S. Provisional Application No. 60/390,498, filed Jun. 21, 2002.

### FIELD OF THE INVENTION

This invention relates to the generation of monoclonal antibodies in a Th1-biased rodent.

### BACKGROUND OF THE INVENTION

Monoclonal antibodies (mAbs) are proven entities for the treatment of various human diseases. In addition, mAbs can represent a powerful tool to gain a better understanding of the immunopathogenesis of various diseases. A standard method for the generation of mAbs consists of fusing myeloma cells with lymph node cells or splenocytes harvested from immunized BALB/c mice (Köhler and Milstein, Nature 256, 495-497 (1975); Köohler and Milstein, Eur. J. Immunol. 6, 511 (1976)). BALB/c mice represent the host of choice for raising mAbs since BALB/c mice are readily available and the immune response in BALB/c mice sensitized with foreign T-dependent antigens is characterized by a polarization of their T-cell derived cytokine production toward a Th2-like phenotype (reviewed in Reiner and Locksley, Ann. Rev. Immunol. 13, 151 (1995)). This Th2-like response is accompanied by the generation of high levels of antigen-specific IgG1 antibodies (Finkelman et al., Ann. Rev. Immunol. 8, 303 (1990)), which correlates with an increase in the frequency of antigen-specific B cell clones.

Advances in transgenic and gene knockout mouse models have provided new ways to make mAbs that are less immunogenic and to study the biology of immune-mediated responses. For example, mice transgenic for human immunoglobulin heavy and light chains can be used to generate human mAbs for therapeutic use. However, transgenic and knockout mice are not from a BALB/c background. Thus, a need exists to generate mAbs in these mice.

Transgenic and knockout mice are generally derived from a C57BL/6 (B6) background (The Jackson Laboratories catalog, 2001). Unfortunately, the B6 genetic background does not represent the optimal immune environment for the generation of mAbs. This is due to the fact that the immune response in antigen-primed B6 mice is Th1-biased, which is characterized by a strong cellular response and a weak humoral response as demonstrated in the classical Th1/Th2 Leishmania major model (Reiner and Locksley, supra). Therefore, the generation of mAbs using B cells harvested from Th1-biased B6 mice can be hindered by the low frequency of antigen-specific B cell clones. Thus, a need exists for methods that skew the immune response in B6 mice toward a Th2-like phenotype. Such methods will result in a more efficient way of generating mAbs due to the higher frequency of antigen-specific B cell clones in Th2-biased hosts.

### BRIEF DESCRIPTION OF THE DRAWINGS

FIG. 1 shows a C57BL/6 mouse immunization schedule.

FIG. 2 is a graph of MCP-1-specific endpoint titers by days in B6 mice primed intramuscularly with plasmid DNA.

FIG. 3 is a graph of MCP-1-specific endpoint titers by days in B6 mice primed intradermally with plasmid DNA.

### SUMMARY OF THE INVENTION

One aspect of the invention is a method for generating monoclonal antibodies in a Th1-biased rodent comprising administering a Th1 antagonist in combination with a Th2 agonist to the rodent; immunizing the rodent with an antigen-encoding nucleic acid; and isolating antigen-specific monoclonal antibodies.

Another aspect of the invention is a method for generating monoclonal antibodies in a Th1-biased rodent comprising the steps of administering a Th1 antagonist in combination with a Th2 agonist to the rodent; immunizing the rodent with an antigen-encoding nucleic acid; administering the antigen without a foreign adjuvant; and isolating antigen-specific monoclonal antibodies.

Yet another aspect of the invention is a method for generating human monoclonal antibodies in a C57BL/6 mouse comprising the steps of administering peglyated IL-4 in combination with an anti-IL-12 monoclonal antibody to the mouse; immunizing the mouse by administering an antigen-encoding nucleic acid intradermally; administering the antigen without a foreign adjuvant intradermally; and isolating antigen-specific monoclonal antibodies.

### DETAILED DESCRIPTION OF THE INVENTION

All publications, including but not limited to patents and patent applications, cited in this specification are herein incorporated by reference as though fully set forth.

The term “in combination with” as used herein and in the claims means that the Th1 antagonists and Th2 agonists described herein can be administered to a rodent together in a mixture, concurrently as single agents or sequentially as single agents in any order.

The present invention provides methods for generating mAbs in a Th1-biased rodent. Administration of a Th1 antagonist in combination with a Th2 agonist to a Th1-biased rodent prior to immunization elicits a Th2-like phenotype that optimizes B-cell proliferation and differentiation. The method of the invention is useful in the generation of antigen-specific IgG1 mabs in Th1-biased rodents such as rats and mice. The mAbs generated by the method of the invention are useful as therapeutic agents, diagnostic agents or research reagents.

Transgenic or gene knockout mice may be used in the method of the invention. For example, mice transgenic for human immunoglobulin genes, such as the HuMab-mouse® (Medarex, Inc., Princeton, N.J.) or the XenoMouse® (Abgenix, Inc., Fremont, Calif.) can be used to generate human antibodies. Gene knockout mice can be used to efficiently generate autologous mAbs against mouse proteins by circumventing immune tolerance of the targeted protein. In particular, mice having a C57BL/6 background may be used.

Agents that interfere with Th1 development are useful as the Th1 antagonists of the invention. Th1 antagonists include, but are not limited to, any antibody, fragment or mimetic, any soluble receptor, fragment or mimetic, any small molecule antagonist, or any combination thereof. In particular, mAbs such as anti-IL-12, anti-IFN-γ or anti-IL-18 can be used as Th1 antagonists. One of ordinary skill in the art could readily determine the amounts of Th1 antagonist to administer. For example, about 0.5mg to about 1 mg of anti-IL-12 per mouse injected intraperitoneally can be used to block Th1 development.

Agents that promote a Th2-type response are useful as the Th2 agonists of the invention. These agents can be nucleic acids or proteins. In particular, IL-4, IL-5 or IL-6 modified to increase half-life can be used. Pegylated IL-4, IL-5 or IL-6 are particularly useful in the method of the invention. See Pepinsky et al., J. Pharm. Exp. Ther. 297, 1059 (2001) and Mori et al., J. Immunol. 164, 5704 (2000). One of ordinary skill in the art could readily determine the amounts of Th2 agonist to administer. For example, about 5μg of pegylated IL-4 per mouse injected intraperitoneally can be used to drive a Th2 immune response.

The timing of adminstration of the Th1 antagonist in combination with the Th2 agonist is preferably pre-immunization, e.g., on the day before immunization (day -1).

After administration of the Th1 antagonist in combination with the Th2 agonist, the rodent is immunized with an antigen-encoding nucleic acid. Immunization of rodents with DNA encoding antigens of interest is a very effective method of generating high-titer antigen-specific IgG antibodies that recognize the native protein target. See Cohen et al., Faseb J. 12, 1611 (1998), Robinson, Int. J. Mol. Med. 4, 549 (1999) and Donnelly et al., Dev. Biol. Stand. 95, 43 (1998). Exemplary plasmid vectors useful to contain the antigen-encoding nucleic acid with or without an adjuvant molecule contain a strong promoter, such as the HCMV immediate early enhancer/promoter or the MHC class I promoter, an intron to enhance processing of the transcript, such as the HCMV immediate early gene intron A, and a polyadenylation (polyA) signal, such as the late SV40 polyA signal. The plasmid can be multicistronic to enable expression of both the antigen and the adjuvant molecule, or multiple plasmids could be used that encode the antigen and adjuvant separately. An exemplary adjuvant is IL-4, others include IL-6, IFN-α, IFN-β and CD40.

It is desirable to administer the antigen-encoding nucleic acid to induce a potent B cell activation and differentiation. Since dendritic cells are the principal cells initiating the immune response after DNA vaccination (Casares et al., J. Exp. Med. 186, 1481 (1997), Akbari et al., J. Exp. Med. 189, 169 (1999) and You et al., Cancer Res. 61, 3704 (2001)), skin Langerhans cells are useful targets for efficient T and B cell priming. Accordingly, the antigen-encoding nucleic acid can be administered intradermally, particularly with weak immunogens. An exemplary immunization schedule is intradermal injection of about 10 μg of antigen-encoding nucleic acid on days 0 and 14. Additional immunization on days 28 and 42 with 10 μg antigen-encoding nucleic acid intradermally may be administered.

After immunization of the rodent, clonal populations of immortalized B cells are prepared by techniques known to the skilled artisan. Antigen-specific mAbs can be identified by screening for binding and/or biological activity toward the antigen of interest by using peptide display libraries or other techniques known to those skilled in the art.

In another embodiment of the invention, rodents are administered a Th1 antagonist in combination with a Th2 agonist, immunized with an antigen-encoding nucleic acid and then administered antigen without foreign adjuvant as a booster. This method is useful in generating high titers of antigen-specific IgG against otherwise weak immunogens. Foreign adjuvant is not required to induce polyclonal antibody response in the method of the invention. An exemplary immunization schedule for this embodiment of the invention is intradermal injection of 10 μg antigen-encoding nucleic acid on days 0 and 14 followed by additional immunization on days 28 and 42 with about 10 μp to about 50 μp purified antigen protein subcutaneously. Accordingly, the method of the invention is particularly useful for the generation of mAbs against those B cell epitopes that might be destroyed in the presence of foreign adjuvant.

The present invention will now be described with reference to the following specific, non-limiting example.

Example 1

### Generation of anti-MCP-1 mAbs in B6 Mice

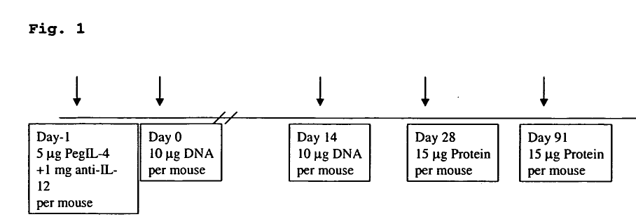

Antibodies were generated in a series of various B6 mouse treatment groups against the weak immunogen MCP-1 (Yoshimura et al., FEBS Lett. 244, 487 (1989)) as shown in Table 1. The immunization schedule used is shown in FIG. 1. In general, 8 to 12 week old C57BL/6 mice were treated with 5μg pegylated murine IL-4 (peg IL-4) and 1 mg neutralizing anti-mouse IL-12 antibody C17.8 (Wysocka et al., Eur. J. Immunol. 25, 672 (1995)) one day prior to the first DNA injection to drive a Th2-like response. At days 0 and 14, 10 μg of MCP-1 plasmid DNA encoding MCP-1 with a HCMV immediate early enhancer/promoter, an HCMV immediate early gene intron A and late SV40 polyA signal were administered to the mice. The mice were boosted at days 28 and 91 with 15 μg MCP-1 protein without any foreign adjuvant. Sera were collected at various time points after protein boosting and levels of MCP-1- and β-galactosidase-specific IgG antibodies were determined by standard ELISA.

Pegylated IL-4 was prepared as follows. 1 mg of murine IL-4 (Research Diagnostics, Inc., Flanders, N.J.) was dissolved in 1 ml of PBS and 10 mg of mPEG(20 K)-SPA (Shearwater Corporation, Huntsville, Ala.) was added to 700 μl of the IL-4 solution. The reaction was incubated at room temperature for 3 hours and quenched with 24 μl of 10 mg/ml Tris in water. Following the addition of the Tris, 600 μl of the reaction mixture was loaded onto a Superose-12 gel filtration column (Amersham Biosciences, Inc., Piscataway, N.J.) having a 24 ml column volume. The column was eluted with PBS at 0.5 ml/min and 1 ml fractions collected. Fractions 26-31 were pooled to give 450 μg of pegylated IL-4.

TABLE 1
Treatment groups

| | Anti-IL-12 + Peg | Plasmid DNA/ | |
|---|---|---|---|
| Groups | IL-4 | Route | Protein Boost |
| 1 (n = 4) | Yes | MCP1-/IM* | MCP-1 (IM) |
| 2 (n = 4) | Yes | MCP-1/ID*-Ears | MCP-1 (IM) |
| 3 (n = 4) | Yes | β-Gal/IM | β-Gal (IM) |
| 4 (n = 4) | No: Rat IgG + Peg | MCP-1/IM | MCP-1 (IM) |
| | alone | | |
| 5 (n = 4) | Nothing | MCP-1/IM | MCP-1 (IM) |

*IM: intramuscular ID: intradermal

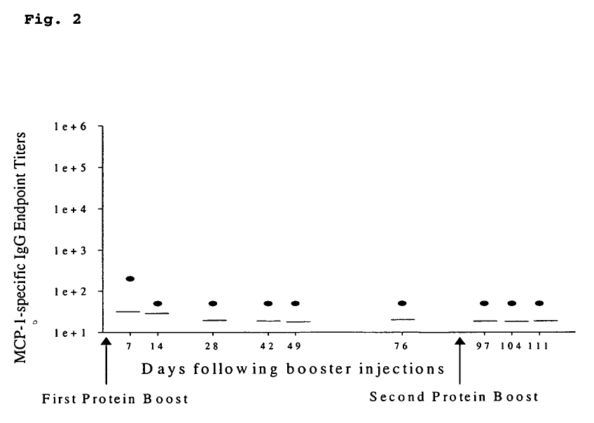

The results indicated that mice primed with DNA using the intramuscular route generated antigen-specific mean IgG titers of 1/100 at all time points tested. FIG. 2 shows the data from treatment group 1 where the horizontal bars represent the mean values of antigen-specific IgG antibodies. Similar results were obtained with mice in treatment groups 3, 4 and 5.

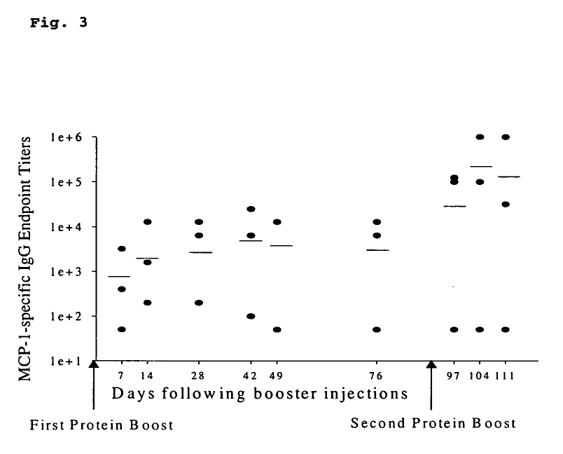

In treatment group 2, 50% ( 2/4) of the mice that were primed with plasmid DNA using the intradermal route also generated antigen-specific IgG antibodies (FIG. 3). The intradermal route resulted in higher levels of antibodies. It was also observed that this approach resulted in the elicitation of a strong B cell memory response as demonstrated by a rapid induction of levels of antigen-specific antibodies following the second protein boost (FIG. 3).

The results also indicated that all of the mAbs generated in the groups treated with peg IL-4 and anti-IL-12 were of the IgG1 isotype.

The present invention now being fully described, it will be apparent to one of ordinary skill in the art that many changes and modifications can be made thereto without departing from the spirit or scope of the appended claims.

1. A method for generating monoclonal antibodies in a Th1-biased rodent comprising the steps of:
a) administering a Th1 antagonist in combination with a Th2 agonist to the rodent;
b) immunizing the rodent with an antigen-encoding nucleic acid; and
c) isolating antigen-specific monoclonal antibodies.

2. A method for generating monoclonal antibodies in a Th1-biased rodent comprising the steps of:
a) administering a Th1 antagonist in combination with a Th2 agonist to the rodent;
b) immunizing the rodent with an antigen-encoding nucleic acid;
d) administering the antigen without a foreign adjuvant; and
d) isolating antigen-specific monoclonal antibodies.

3. The method of claim 1 or 2 wherein the rodent is a mouse.

4. The method of claim 3 wherein the mouse is a C57BL/6 mouse.

5. The method of claim 1 or 2 wherein the rodent is a rat.

6. The method of claim 1 or 2 wherein the Th1 antagonist is a nucleic acid or a protein.

7. The method of claim 6 wherein the Th1 antagonist is a monoclonal antibody that interferes with Th1 development.

8. The method of claim 7 wherein the Th1 antagonist is an anti-IL-12, anti-IFN-γ or anti-IL-18 antibody.

9. The method of claim 1 or 2 wherein the Th2 agonist is modified to extend its half-life.

10. The method of claim 9 wherein the Th2 agonist is pegylated IL-4, pegylated IL-5 or pegylated IL-6.

11. The method of claim 1 or 2 wherein the antigen-encoding nucleic acid is administered intradermally.

12. The method of claim 1 or 2 wherein the monoclonal antibodies are human.

13. The method of claim 2 wherein the antigen is administered intradermally.

14. A method for generating human monoclonal antibodies in a C57BL/6 mouse comprising the steps of:
a) administering peglyated IL-4 in combination with an anti-IL-12 monoclonal antibody to the mouse;
b) immunizing the mouse by administering an antigen-encoding nucleic acid intradermally;
c) administering the antigen without a foreign adjuvant intradermally; and
d) isolating antigen-specific monoclonal antibodies.

15. A method for generating human monoclonal antibodies in a C57BL/6 mouse comprising the steps of:
a) administering peglyated IL-4 in combination with an anti-IL-12 monoclonal antibody to the mouse;
b) immunizing the mouse by administering an antigen-encoding nucleic acid intradermally; and
c) isolating antigen-specific monoclonal antibodies.
